(19) United States
(12) Patent Application Publication
(10) Pub. No.: US 2005/0150801 A1
(43) Pub. Date: Jul. 14, 2005

(54) Package having an expansion mechanism

(75) Inventor: Darold Dean Tippey, Neenah, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/758,640

(22) Filed: Jan. 14, 2004

(51) Int. Cl. A61L 15/00

(52) U.S. Cl. 206/440; 206/494; 206/449

(57) ABSTRACT

A package is disclosed having a first sleeve and a second sleeve. Each sleeve has at least one side wall and is closed at one end by an end wall. The second sleeve is sized so as to be capable of moving in a telescopic fashion on the first sleeve. An array of compressible articles is enclosed between the first and second sleeves which form an enclosed package. Each article has at least one planar surface aligned substantially parallel to at least one of the end walls and is held in compression in a direction that is substantially perpendicular to the planar surface. The package also has an attachment and release member securing the first and second sleeves together after the articles have been compressed.

### BACKGROUND OF THE INVENTION

Today many manufacturers are using compressed packaging to reduce the size and volume of their packages. A smaller package reduces distribution and shipping costs while providing the same number of products to the consumer. A smaller package also requires less shelf space at a retail store which means that additional packages can be stocked and displayed for sale in a similar size area as was used for the larger size packages. Compressed packaging works especially well for absorbent articles, such as diapers, training pants, adult incontinent garments, feminine napkins, adult incontinent pads, wet wipes, facial tissue, etc. which normally contain air. Absorbent articles can be compressed to eliminate air between adjacent articles as well as to flatten or downsize the actual article. One trade off with a more densely compressed package is that it is usually harder to withdraw the first few articles from the opened package.

Various package designs have been tried to alleviate this problem but most have had only modest in-use success. For absorbent articles in particular, the articles are designed for various age groups. For example, diapers designed for infants and young children normally require a parent or caregiver to remove the article from the package while at the same time restraining the infant or child. This means that sometimes the parent or caregiver has only one hand available to remove the diaper from the package. As for older adults using incontinent pads and undergarments, many suffer from arthritis in their hands and/or poor eyesight and it may be difficult for them to extract a single article from a highly compressed package.

Now a package has been developed that utilizes a unique design that allows the package and articles retained therein to be compressed and still provides for easy removal of the first few articles by the ultimate consumer. The design incorporates an expansion mechanism which allows the package to increase in size and volume before the articles are withdrawn.

### SUMMARY OF THE INVENTION

Briefly, this invention relates to a package having an expansion mechanism to allow for easy removal of the first few articles from a compressed package. The package includes a first sleeve and a second sleeve. Each sleeve has at least one side wall and is closed at one end by an end wall. The second sleeve is sized so as to be capable of moving in a telescopic fashion on the first sleeve. An array of compressible articles is enclosed between the first and second sleeves which form an enclosed package. Each article has at least one planar surface aligned substantially parallel to at least one of the end walls and is held in compression in a direction that is substantially perpendicular to the planar surface. The package also has an attachment and release member securing the first and second sleeves together after the articles have been compressed. The attachment and release member is capable of being removed whereby the second sleeve will telescopically move upward relative to the first sleeve as the articles expand so as to increase the volume of the package and allow the articles to be individually removed.

### BRIEF DESCRIPTION OF THE DRAWINGS

FIG. 1 is a perspective view of a package having an expansion mechanism.

FIG. 2 is a cross-sectional view of the first sleeve showing an array of compressible articles, in a non-compressed condition, such that they extend beyond the upper edge of the side walls.

FIG. 3 is a cross-sectional view of an array of compressible articles, in a non-compressed condition, positioned between the first and second sleeves.

FIG. 4 is a cross-sectional view of the package shown in FIG. 1 taken along line 4-4.

FIG. 5 is a top view of the package shown in FIG. 1 after the flap has been removed, thereby creating an opening through which the articles can be individually withdrawn.

FIG. 6 is a top view of an alternative embodiment depicting a package wherein the first and second sleeves have a square cross-sectional configuration.

FIG. 7 is a cross-sectional view of the package shown in FIG. 6 taken along line 7-7 and depicting the first sleeve as having a side wall that has a predetermined height that is greater than the predetermined height of the side wall of the second sleeve.

### DETAILED DESCRIPTION

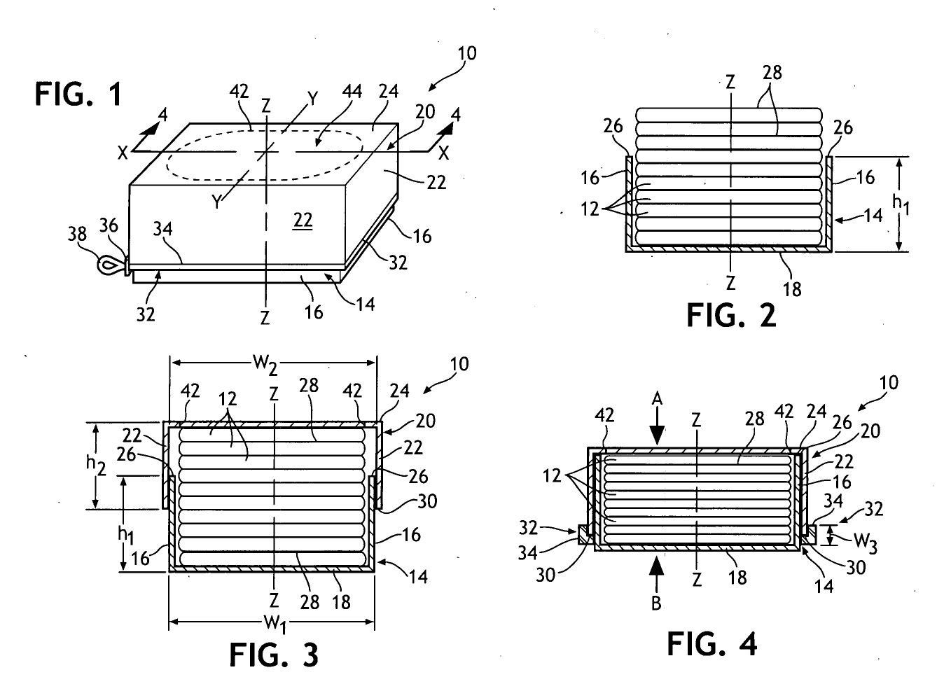

Referring to FIGS. 1-4, a package 10 is shown which is adapted to retain an array of compressible articles 12. By “compressible” is meant that the articles 12 are capable of being compressed in one or more directions. By “compress” it is meant to press or squeeze together, to shorten or condense, to flatten laterally or lengthwise. For example, the volume of the package 10 can be reduced. The package 10 could be compressed by applying pressure in opposite directions to the package 10, as indicated by the two arrows, denoted A and B, in FIG. 4. The array of compressible articles 12 can be absorbent articles, for example, disposable absorbent articles. Such articles 12 can include infant diapers, training pants, and adult incontinent garments including briefs, pants and refastenable undergarments. The compressible articles 12 can also be incontinent pads, feminine sanitary napkins, pantyliners, menstrual pants, wet wipes, facial tissue, paper towels, paper napkins, or any other absorbent article known to those skilled in the art. Furthermore, the compressible articles 12 can be non-absorbent articles that are capable of being compressed. Examples of non-absorbent articles include clothing, some foods, medicines, some sporting goods, etc.

Normally, from between 1 to about 200 articles 12 can be retained in a single package 10. Today, package counts of 1, 2, 3,4, 6, 8, 10,12, 18, 20 24, 50, 75,100, etc., exist. The exact number of articles 12 retained in a package 10 can vary depending on the size and shape of the articles themselves, the cost of the articles, the retail outlet where the articles will be sold, the frequency of how fast the articles are used, etc. Desirably, a package 10 containing disposable absorbent articles will contain at least six articles 12. More desirably, a package 10 containing disposable absorbent articles will contain at least twelve articles 12. Most desirably, a package 10 containing disposable absorbent articles will contain less than about 60 articles 12.

When the articles 12 are compressed, air located in and/or between adjacent articles can be squeezed, expelled out or drawn out by pressure or vacuum, to make a smaller, denser package. Such compressed articles 12 are capable of expanding or enlarging back to or towards their initial non-compressed state once the compressive force is removed, such as when the volume of the package 10 is increased or when the package 10 is opened.

Referring to FIG. 1, the package 10 is shown having a longitudinal axis X-X, a transverse axis Y-Y and a vertical axis Z-Z. The following discussion will describe the package 10 in terms of having a bottom wall, a top wall and side walls so as to follow the orientation of the package 10, as shown in FIG. 1. If one wished to rotate the package 10, for example ninety degrees, then the top wall would become a side wall, one of the side walls would become the bottom wall, etc. It should be evident to one skilled in the art that the subject matter of this invention should not be limited to the initial orientation of the package 10.

The package 10 includes a first sleeve 14 which includes at least one side wall 16. Four side walls 16 are present in the package 10, with two of the side walls 16 being visible in FIG. 1. If the package had a cylindrical profile, such as a tube, a single side wall would be present. If the package had a triangular top surface then three side walls would be present. For a box type package having a square or rectangular top surface, four side walls would be utilized. Each of the side walls 16 has a predetermined height h₁ and one end of each side wall 16 is attached to a first wall 18, see FIGS. 2-4. The first wall 18 closes off one end of the first sleeve 14 and produces a hollow container having a bottom wall but no top wall. The height h₁ of each of the side walls 16 can be of any desired dimension. However, for packaging compressible absorbent articles, such as feminine pads, pantyliners or adult incontinence pads, it is envisioned that the height h₁ of each side wall 16 will range from between about 1 inch (about 2.54 centimeters (cm)) to about 24 inches (about 61 cm). Desirably, the height h₁ of each side wall 16 will range from about 2 inches (about 5 cm) to about 18 inches (about 46 cm). More desirably, the height h₁ of each side wall 16 will range from about 3 inches (about 7.6 cm) to about 12 inches (about 30.5 cm). Most desirably, the height h₁ of each side wall 16 will be less than about 10 inches (about 25.4 cm).

The first sleeve 14 can be constructed from various materials, including a flexible material, a pliable material, a semi-rigid material, a stiff material or a combination of such materials. Flexible and pliable materials include paper, thermoplastic films including polyethylene films and polypropylene films, plastic, plastic film, plastic laminates, a blend of two or more plastic materials, a blend of paper and a plastic material, a non-woven, or a cloth material such as cotton, nylon, rayon, polyester, etc. Other flexible materials known to those skilled in the art can also be used. Alternatively, one or more walls 16 or 18 of the first sleeve 14 can be semi-rigid in structure. By “semi-rigid” it is meant a material that may be stiff in at least one direction but can be easily bent or distorted in one or more directions. Examples of some semi-rigid materials include different grades of cardboard, paper board, various plastic sheets, blended films, laminates, a thin wood veneer, etc. Still further, the first sleeve 14 can be constructed from a “rigid material.” A “rigid material” can include wood, a thick plastic sheet, a stiff synthetic material, glass, etc.

It should be noted that one portion of the first sleeve 14 can be formed from one material and remaining portions of the first sleeve 14 can be formed from a different material.

For some applications, it may be desirable to construct the first sleeve 14 from a material that can itself be compressed after a plurality of compressible articles 12 are inserted therein. The material should be dimensionally stable once the package 10 is compressed and sealed and therefore can maintain its dimensions while subjected to pressure from within the package 10 until the package 10 is allowed to expand or is opened. Polypropylene and polyethylene films, as well as a laminate formed therefrom are pliable materials that are commercially available today that can be compressed. When a plastic film is utilized, it can have almost any thickness but a thickness of less than about 5 millimeters (mm) is useful for most packages. A film material having a thickness of between about 1 to about 5 mm can be very cost effective, especially when large quantities of packages need to be manufactured. Flexible plastic bags and packages constructed from a thin sheet of material are very advantageous to use since they are compressible and do not have rigid corners.

Referring to FIGS. 1, 3 and 4, the package 10 also includes a second sleeve 20 which includes at least one side wall 22. Four side walls 22 are present in the package 10 with two of the side walls 22 being visible in FIG. 1. Each of the side walls 22 has a predetermined height h₂ and one end of each side wall 22 is attached to a second wall 24. The second wall 24 closes off one end of the second sleeve 20 and produces a hollow container having a top wall but no bottom wall. The height h₂ of each of the side walls 22 can be less than, equal to or greater than the height h₁ of each of the side walls 16. Desirably, the height h₂ of each of the side walls 22 is approximately equal to or less than the height h₁ of each of the side walls 16. Most desirably, the height h₂ of each of the side walls 22 is less than the height h₁ of each of the side walls 16. By adjusting the height relationship between h₁ and h₂, a very aesthetically pleasing package can be designed. However, the height h₂ of each of the side walls 22 should be of sufficient length such that the side walls 22 of the second sleeve 20 overlap at least a portion of the side walls 16 of the first sleeve 14 before the array of articles 12 positioned therebetween are compressed. This feature will simplify the compression step.

The height h₂ of each of the side walls 22 of the second sleeve 20 can be of any desired dimension. However, for packaging compressible absorbent articles, such as feminine pads, pantyliners or adult incontinence pads, it is envisioned that the height h₂ of each side wall 22 range from between about 1 inch (about 2.54 centimeters (cm)) to about 24 inches (about 61 cm). Desirably, the height h₂ of each side wall 22 will range from about 2 inches (about 5 cm) to about 18 inches (about 46 cm). More desirably, the height h₂ of each side wall 22 will range from about 3 inches (about 7.6 cm) to about 12 inches (about 30.5 cm). Most desirably, the height h₂ of each side wall 22 will be less than about 10 inches (about 25.4 cm).

It should be noted that the second sleeve 20 can be formed from any of the materials described above in reference to the first sleeve 14.

Referring now to FIG. 3, the second sleeve 20 is sized so as to be capable of moving in a telescopic fashion on the first sleeve 14. Assuming that the first and second sleeves, 14 and 20 respectively, have the same geometrical shape, the first sleeve 14 will be slightly smaller in cross-sectional area than the second sleeve 20. For example, in FIG. 3, the first sleeve 14 has an outside width w₁ which is slightly less than the interior width w₂ of the second sleeve 20. This size difference will allow the second sleeve 20 to move or slide up and/or down relative to the first sleeve 14. The exact size dimension between the first and second sleeves, 14 and 20 respectively, can vary. However, in order to manufacture a functional package 10, the dimensional difference between w₁ and w₂ should range from between about 1 mm to about 15 mm. Desirably, the dimensional difference between w₁ and w₂ should range from between about 2 mm to about 10 mm. More desirably, the dimensional difference between w₁ and w₂ should range from between about 3 mm to about 5 mm. The exact dimensional difference will be dependent upon various factors, including but not limited to: the types of materials from which the first and second sleeves, 14 and 20 respectively, are constructed from; the height of each sleeve; the cross-sectional shape of each sleeve; the presence of a finish on the sleeves; the coarseness of each sleeve; etc.

Referring again to FIGS. 2-4, the package 10 is designed to contain and enclose the array of articles 12 enclosed between said first and second sleeves, 14 and 20 respectively. This is accomplished by positioning or stacking non-compressed articles 12 in the first sleeve 14, as is shown in FIG. 2. The non-compressed articles 12 will fill the first sleeve 14 and extend beyond an upper edge 26 of the side walls 16. The distance of the extension beyond the upper edge 26 of the side walls 16 can vary depending upon the type of articles being packaged, the thickness of each article, the number of articles one wishes to retain in the package 10, and the amount one wishes to compress the articles. In FIG. 2, one will notice that the first wall 18 functions as the bottom wall of the package 10. Looking now at FIG. 3, one can clearly see that the second sleeve 20 is positioned over the non-compressed articles 12 with the second wall 24 functioning as the top wall of the package 10. The side walls 22 of the second sleeve 20 will enclose and confine the non-compressed articles 12 that extend upward out of the first sleeve 14. The side walls 22 of the second sleeve 20 will also overlap a portion of the side walls 16 of the first sleeve 14. At this time, the array of non-compressed articles 12 will be vertically stacked within the first and second sleeves, 14 and 20 respectively.

Each of the articles 12 has at least one planar surface 28 aligned substantially parallel to at least one of said first (bottom) and second (top) walls, 18 and 24 respectively. By “planar” is meant a relatively flat surface having two dimensions, for example, a length and a width. For example, if the article 12 is an adult incontinent pad, the upper and lower flat surfaces of the pad would qualify as the planar surfaces 28. The array of articles 12 will be held in compression in a direction that is substantially perpendicular to the planar surface 28. The articles 12 can be compressed by applying a force to the top and bottom walls, 24 and 18 respectively, see FIG. 4, so as to cause the second sleeve 20 to move downward in a telescopic fashion over the first sleeve 14. The pressure is denoted by the two arrows A and B. As the second sleeve 20 moves downward, a lower edge 30 of the side walls 22 will approach the bottom wall 18 of the first sleeve 14. For best results, the lower edge 30 of the side walls 22 should stop short of the bottom wall 18. The reason for this slight offset is to allow an attachment and release member 32 to be secured to both of the first and second sleeves, 14 and 20 respectively. In order to reduce the amount of material needed to form the first and second sleeves, 14 and 20 respectively, the side walls 16 of the first sleeve 14 should be sized to a length that will allow the side walls 22 of the second sleeve 20 to be positioned in the desired location when the upper edge 26 of the side walls 16 contact the inside surface of the top wall 24. This will assure that the array of articles 12 will be compressed to the maximum amount possible without deforming or buckling the side walls 16 or 22.

Still referring to FIGS. 1 and 4, the attachment and release member 32 is designed to bridge across the first and second sleeves, 14 and 20 respectively, once the articles 12 have been compressed and the second sleeve 20 has sufficiently overlapped a major portion of the first sleeve 14. The attachment and release member 32 is designed to be easily removed by the ultimate customer when they are ready to use the product. The attachment and release member 32 can be secured or attached to the package 10 by ultrasonics, by adhesive, by using microwave energy, by heat, by pressure, by a combination of heat and pressure, or by other ways known to those skilled in the art.

The attachment and release member 32 can be in the form of an elongated strip 34 that encircles the perimeter of the package 10. The width and length dimensions of the strip 34 can vary to accommodate the size and configuration of the package 10. The attachment and release member 32 is designed to be easily removed so that the array of compressed articles 12 can expand and allow the second sleeve 20 to telescopically move upward relative to said first sleeve 14. Until the attachment and release member 32 is removed, the second sleeve 20 will be held secure to the first sleeve 14 and the compressed articles 12 will not be able to expand. The upward movement of the second sleeve 20 is important for it allows the volume of the package 10 to increase to accommodate the expansion of the compressed articles 12. The volume of the package 10 can be increased from between about 20% to about 100% once the attachment and release member 32 is removed. Desirably, the volume of the package 10 can be increased from between about 25% to about 75% once the attachment and release member 32 is removed. Most desirably, the volume of the package 10 can be increased by at least about 50% once the attachment and release member 32 is removed. Once the articles 12 have expanded and the volume of the package 10 has significantly increased, the consumer can easily remove each article 12 individually through an opening formed in the package 10. Up until now, it has been difficult to individually withdraw single articles from a compressed package that does not have an expansion mechanism. Normally, when one tries to withdraw the first compressed article from a package, without an expansion mechanism, two or more articles will be dispensed. This occurs because the compressed articles tend to stick together until sufficient void volume is present in the package such that the articles can sufficiently separate from one another.

Still referring to FIGS. 1 and 4, the attachment and release member 32 is normally formed from a material that is different from the material from which the first and second sleeves, 14 and 20 respectively, are formed. The reason for this is that the attachment and release member 32 must posses different properties so as to be capable of functioning as both an attachment member and release member. However, in some applications, the attachment and release member 32 can be formed from a material that is identical to or is the same material as was used to construct the package 10.

In FIGS. 1 and 4, the attachment and release member 32 is shown as a separate band or strip 34 encircling the entire perimeter of the package 10. The band or strip 34 is designed to be completely torn or separated from the package 10 so as to allow the second sleeve 20 to freely move relative to the first sleeve 14. The band or strip 34 has a width w₃, see FIG. 4, which can vary to suit one's particular needs. The width w₃ of the band or strip 34 can range from between about 1 mm to about 60 millimeters. Desirably, the width w₃ of the band or strip 34 is less than about 15 mm. More desirably, the width w₃ of the band or strip 34 is less than about 10 mm. Most desirably, the width w₃ of the strip 34 is less than about 5 mm. A narrow band or strip 34 may be less costly than a wider strip.

Returning again to FIG. 1, the attachment and release member 32 is shown as a strip 34 having a terminal end 36 and having a pull tab 38 secured or connected to the terminal end 36. The pull tab 38 can be sized and shaped as a finger ring or have some other geometrical configuration. The dimensions of the pull tab 38 can be designed to easily fit between a persons thumb and index finger so that it can be easily grasped. The pull tab 38 can be a solid piece of material or be in the shape of a ring having an opening formed therein. The function of the pull tab 38 is to provide an easy means for the consumer to remove the strip 34 from around the perimeter of the package 10.

The attachment and release member 32 can be formed from a heat shrinkable material, from a material that can form a seal, from a breakable material, from a frangible material, from a tear strip or from a zip lock strip. The attachment and release member 32 can also be formed as a zipper or a VELCRO® attachment. VELCRO® is a registered trademark of Velcro USA, Inc. having an office at 406 Brown Avenue, Manchester, N.H. 03103. The attachment and release member 32 will extend across the juncture of the first and second sleeves, 14 and 20 respectively, and will be securely adhered to the outside surface of each sleeve, 14 and 20. In FIG. 1, the attachment and release member 32 is depicted as a releasable tear strip 34 formed near the bottom wall 18. By forming the attachment and release member 32 near the bottom wall 18 (see FIG. 4) of the package 10, one can be assured that removal of the strip 34 will not interfere or deface the graphics which may appear on the outside surface of the package 10.

It should be noted that the attachment and release member 32 can be a single continuous strip 34 or be a combination of two strips. One benefit of forming the attachment and release member 32 as a single tearable strip is that when the tearable strip is broken, a clean break occurs. This is an important aesthetic feature that many consumers like. Another benefit of using a single tear strip 34 is that it is easy to dispose of. It is also possible to color or tint the attachment and release member 32 so as to make it more visible relative to other color or graphics that may appear on the package 10. A visually distinctive attachment and release member 32 can catch the attention of the consumer's eyes and aid them in properly opening the package 10.

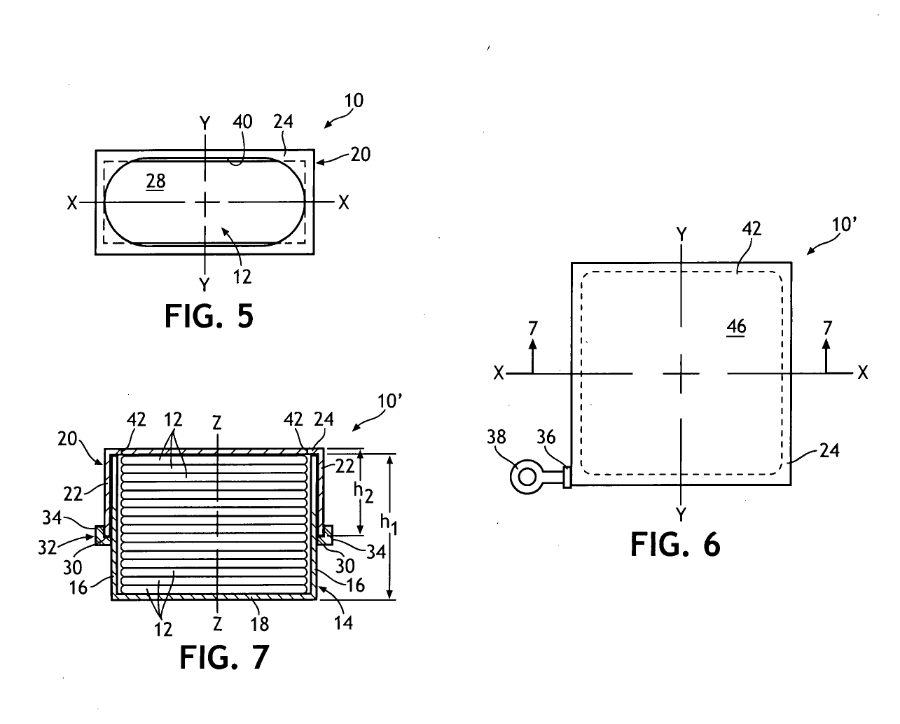

Referring now to FIG. 5, the package 10 requires an opening 40 formed in at least one of the walls 16, 18, 22 or 24 so that the articles 12 retained therein can be individually removed or withdrawn. The opening 40 can vary in shape, size, configuration and placement on the package 10. For example, the opening 40 can be formed in the top wall 24, in one or more of the side walls 16 or 22, or be formed in the bottom wall 18. It is also possible to form the opening 40 in two or more adjacent walls, for example at the intersection of the top wall 24 and a side wall 22. The shape of the opening 40 can be almost any geometrical configuration one desires. The size of the articles 12 can influence the actual shape that is utilized. In FIG. 5, the opening 40 is formed in the top wall 24 and has a generally oval shape. The size of the opening 40 is slightly smaller than the planar surface 28 of each article 12. Desirably, the size of the opening 40 will be sufficiently large to allow the consumer to reach in with his or her fingers and thumb and grab the topmost article 12. This is important because as more articles 12 are removed from the package 10, the subsequent topmost article 12 may be located below the height h₁ of the first sleeve 14. Unless the package 10 has a lifting mechanism, the articles 12 will be located away from the opening 40 and closer to the bottom wall 18. If the opening 40 is too small in size, it may be difficult for the consumer to remove the last few articles 12.

Returning to FIG. 1, the package 10 is depicted having a top wall 24 with a perforation line 42 formed therein. The perforation line 42 is a series of holes or slots punched or bored through the top wall 24. The perforation line 42 circumscribes a removable flap 44. The removable flap 44 is shown having an oval configuration although it could have any desired geometrical shape. The perforation line 42 is depicted as a continuous line but it could be formed as a non-continuous line, if desired. The perforation line 42 is designed to be easily broken by applying pressure onto the flap 44 adjacent to the perforation line 42. Such force will break at least a portion of the perforation line 42 thereby allowing the flap 44 to be grasped by the consumer and be completely torn or severed from the package 10. Once separated from the package 10, the flap 44 can be discarded. After the flap 44 is separated from the package 10, the opening 40, depicted in FIG. 5 will be present. The opening 40, as explained above, should be sufficiently large to allow easy withdrawal of the retained articles 12 one at a time.

Referring now to FIGS. 6 and 7, an alternative embodiment of a package 10′ is depicted. The package 10′ is similar in construction to the package 10 except for a couple of differences. First, the package 10′ has a square cross-sectional configuration instead of a rectangular cross-sectional configuration. Sometimes, the shape of the articles 12 fit better in a square package 10′. A second difference in the package 10′ is that the predetermined height h₁ of the side walls 16 of the first sleeve 14 is greater than that shown in FIGS. 1-4. This means that the package 10′ can be taller than the package 10. A third difference is that in the package 10′, the predetermined height h₁ of the side walls 16 of the first sleeve 14 is much greater than the predetermined height h₂ of the side wall 22 of the second sleeve 20. Therefore, the distance between the lower edge 30 of the side walls 22 of the second sleeve 20 and the bottom wall 18 of the first sleeve 14 will be greater than that shown in FIG. 4. This size difference between the side walls 16 and 22 will cause the attachment and release member 32 to be located farther away from the bottom wall 18 in package 10′. By locating the attachment and release member 32 higher up on the side wall 16, it may be easy for the consumer to visually notice it.

The package 10′ is also different from the package 10 in that it has a perforation line 42 that circumscribes a removable flap 46 having a generally square profile with rounded corners. The removable flap 46 is sized slightly less than the area of the top wall 24 which will make it very easy to withdraw articles 12 from the package 10′.

While the invention has been described in conjunction with several specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

1. A package comprising:
a) a first sleeve including at least one side wall having a predetermined height and being closed at one end by a first wall;
b) a second sleeve including at least one side wall having a predetermined height and being closed at one end by a second wall, said second sleeve being sized so as to be capable of moving in a telescopic fashion on said first sleeve;
c) an array of compressible articles enclosed between said first and second sleeves with said first wall forming a bottom wall of a package and said second wall forming a top wall of said package, said articles having at least one planar surface aligned substantially parallel to at least one of said first and second walls and being held in compression in a direction that is substantially perpendicular to said planar surface; and
d) an attachment and release member securing said first sleeve to said second sleeve after said array of articles have been compressed, said attachment and release member capable of being removed whereby said second sleeve will telescopically move upward relative to said first sleeve as said articles expand so as to increase the volume of said package and allow said articles to be individually removed through an opening formed in said package.

2. The package of claim 1 wherein said first and second sleeves have a rectangular cross-sectional configuration.

3. The package of claim 1 wherein said first and second sleeves have a square cross-sectional configuration.

4. The package of claim 1 wherein said predetermined height of said side wall of said first sleeve is equal to said predetermined height of said side wall of said second sleeve.

5. The package of claim 1 wherein said predetermined height of said side wall of said first sleeve is greater than said predetermined height of said side wall of said second sleeve.

6. The package of claim I wherein said top wall has a perforation line formed therein that circumscribes a removable flap.

7. The package of claim 1 wherein at least one of said first and second sleeves is constructed from a semi-rigid material.

8. The package of claim 7 wherein at least one of said first and second sleeves is constructed of cardboard.

9. The package of claim 1 wherein at least one of said first and second sleeves is constructed from a pliable material.

10. A package comprising:
a) a first sleeve including at least one side wall having a predetermined height and being closed at one end by a first wall;
b) a second sleeve including at least one side wall having a predetermined height and being closed at one end by a second wall, said second sleeve being sized so as to be capable of moving in a telescopic fashion on said first sleeve;
c) an opening formed in said second wall;
d) an array of compressible articles enclosed between said first and second sleeves with said first wall forming a bottom wall of a package and said second wall forming a top wall of said package, said articles having at least one planar surface aligned substantially parallel to at least one of said first and second walls and being held in compression in a direction that is substantially perpendicular to said planar surface; and
e) an attachment and release member securing said first sleeve to said second sleeve after said array of articles have been compressed, said attachment and release member capable of being removed whereby said second sleeve will telescopically move upward relative to said first sleeve as said articles expand so as to increase the volume of said package and allow said articles to be individually removed through said opening.

11. The package of claim 10 wherein said opening is sized to be slightly smaller than said planar surface of each article.

12. The package of claim 11 wherein said opening has an oval configuration.

13. The package of claim 10 wherein said array of compressible articles includes at least six absorbent articles.

14. The package of claim 10 wherein said predetermined height of said side wall of said first sleeve is equal to said predetermined height of said side wall of said second sleeve.

15. The package of claim 10 wherein said predetermined height of said side wall of said first sleeve is greater than said predetermined height of said side wall of said second sleeve.

16. A package comprising:
a) a first sleeve including at least one side wall having a predetermined height and being dosed at one end by a first wall;
b) a second sleeve including at least one side wall having a predetermined height and being closed at one end by a second wall, said second sleeve being sized so as to be capable of moving in a telescopic fashion on said first sleeve;
c) a perforation line formed in said second wall that circumscribes a flap that can be removed to form an opening In said second wall;
d) an array of compressible articles enclosed between said first and second sleeves with said first wall forming a bottom wall of a package and said second wall forming a top wall of said package, said articles having at least one planar surface aligned substantially parallel to at least one of said first and second walls and being held in compression in a direction that is substantially perpendicular to said planar surface; and
e) an attachment and release member securing said first sleeve to said second sleeve after said array of articles have been compressed, said attachment and release member capable of being removed whereby said second sleeve will telescopically move upward relative to said first sleeve as said articles expand so as to increase the volume of said package and allow said articles to be individually removed.

17. The package of claim 16 wherein a pull tab is attached to said attachment and release member.

18. The package of claim 16 wherein said array of compressible articles includes at least twelve absorbent articles.

19. The package of claim 16 wherein said side wall of said first sleeve has a height of less than about 10 inches.

20. The package of claim 16 wherein the volume of said package can be increased by at least about 50% after said attachment and release member is removed.
